(19) United States
(12) Patent Application Publication
(10) Pub. No.: US 2017/0301094 A1
(43) Pub. Date: Oct. 19, 2017

(54) IMAGE COMPOUNDING BASED ON IMAGE INFORMATION

(71) Applicant: KONINKLIJKE PHILIPS N.V., EINDHOVEN (NL)

(72) Inventors: Francois Guy Gerard Marie Vignon, Croton on Hudson, NY (US); William Hou, Briarcliff Manor, NY (US); Jean-Luc Robert, Cambridge, MA (US); Emil George Radulescu, Ossining, NY (US); Ji Cao, Lynwood, WA (US)

(21) Appl. No.: 15/102,907

(22) PCT Filed: Dec. 8, 2014
(86) PCT No.: PCT/IB2014/066691

§ 371 Date: Jun. 9, 2016

Related U.S. Application Data
(60) Provisional application No. 61/913,452, filed on Dec. 9, 2013.

(51) Int. Cl. G06T 7/11; A61B 8/08; G06T 5/20

(52) U.S. Cl. CPC G06T 7/11; G06T 5/20; A61B 8/5269; A61B 8/5253; G06T 2207/10132; G06T 2207/20024

(57) ABSTRACT

An image compounding apparatus acquires, via ultrasound, pixel-based images (126-130) of a region of interest for, by compounding, forming a composite image of the region. The image includes composite pixels (191) that spatially correspond respectively to pixels of the images. Further included is a pixel processor for beamforming with respect to a pixel from among the pixels, and for assessing, with respect to the composite pixel and from the data acquired (146), amounts of local information content of respective ones of the images. The processor determines, based on the assessment, weights for respective application, in the forming, to the pixels, of the images, that spatially correspond to the composite pixel. In some embodiments, the assessing commences operating on the data no later than upon the beamforming. In some embodiments, brightness values are assigned to the spatially corresponding pixels; and, in spatial correspondence, the maximum and the mean values are determined. They are then utilized in weighting the compounding.

### FIELD OF THE INVENTION

The present invention relates to weighting for image compounding and, more particularly, to adaptation that weights according to local image content.

### BACKGROUND OF THE INVENTION

Compounding in ultrasound consists of imaging the same medium with different insonation parameters and averaging the resulting views.

For example, in spatial compounding the medium is imaged at view angles. This results in decreased speckle variance and increased visibility of plate-like scatterers (boundaries) along with other image quality improvements. The averaging reduces noise and improves image quality, because, although the views have respectively different noise patterns, they depict in the context of medical ultrasound similar anatomical features. In addition, certain structures are visible, or more visible, only at certain angles and can be enhanced through spatial compounding.

Since, however, the speed of sound varies by as much as 14% in soft tissue, a slight positioning mismatch of structures is present for the different views. The compounding then causes blurring.

Spatial compounding may be varied adaptively to improve the outcome.

Tran et al. realigns the views using a non-rigid registration that makes use of edge detection as an image metric. See Tran et al, SPIE 2008, “Adaptive Spatial Compounding for Improving Ultrasound Images of the Epidural Space on Human Subjects.”

### SUMMARY OF THE INVENTION

What is proposed herein below is directed to addressing one or more of the above concerns.

Spatial compounding is the default imaging mode on most commercial ultrasound platforms for linear and curvilinear arrays.

However, simply averaging the views is, as mentioned above, not an optimal process: speed of sound errors result in mis-registration of the views leading to a blurry aspect of the images especially at great depths; the sidelobes of the point-spread functions at different view angles are averaged resulting in increased smearing of tissue into cysts; grating lobes from the angled views corrupt the image; and sometimes structures that are only visible at a given angle do not get such a high visibility enhancement because the best sub-view is averaged with other, sub-optimal ones. All these effects result in a decreased contrast of the compounded view with respect to single-view images.

Channel data contain much more information than B-mode images obtained after ultrasound receive beamforming. Therefore, channel-data-based beamforming techniques can provide better sensitivity and/or specificity. Locally adaptive compounding based on a signal metric, and optionally an image metric in addition, can therefore be used to advantage.

In accordance with what is proposed herein, multiple pixel-based images of a region of interest are acquired by ultrasound. They are acquired for, by compounding, forming an image comprising a plurality of pixels that spatially correspond respectively to pixels of the multiple images. Beamforming is performed with respect to a pixel from among the plurality of pixels. Based on the data acquired, an assessment is made, with respect to that pixel, on the amounts of local information content of respective ones of the multiple images. Based on the assessment, weights are determined for respective application, in the forming of the image, to the pixels, of the multiple images, that spatially correspond to that pixel. The assessing commences operating on the data no later than upon the beamforming.

The above steps can be carried out by a locally-adaptive pixel-compounding imaging apparatus. For such a device, a computer readable medium or alternatively a transitory, propagating signal is part or what is proposed herein. A computer program embodied within a computer readable medium as described below, or, alternatively, embodied within a transistory, propagating signal, has instructions executable by a processor for performing the above-specified steps.

In another version, a locally-adaptive pixel-compounding medical imaging apparatus includes an imaging acquisition module configured for, via ultrasound, acquiring multiple pixel-based images of a body-tissue region of interest for, by compounding, forming an image of the region. The image includes pixels that spatially correspond respectively to pixels of the images. The apparatus also includes a pixel processor configured for, based on the data acquired, assessing, with respect to a pixel of the image to be formed, amounts of local information content of respective ones of said images. It is also configured for, based on the assessment, determining weights for respective application, in the forming, to the pixels, of the images, that spatially correspond to that pixel. It further features a pixel compounder configured for, by the applying, creating weighted pixels and for summing the weighted pixels to yield a weighted average of the pixels that spatially correspond to the pixel of the image being formed.

Details of the novel, locally-adaptive pixel-compounding are disclosed below with the aid of the following drawing, which is not drawn to scale, and the following formula sheet and flow charts.

### BRIEF DESCRIPTION OF THE DRAWINGS

FIG. 1 is a schematic diagram of a locally-adaptive pixel-compounding apparatus in accordance with the present invention;

FIG. 2 is a set of mathematical definitions and relationships in accordance with the present invention; and

FIGS. 3A-3C are flow charts of a signal-metric-based, locally-adaptive pixel-compounding process in accordance with the present invention.

### DETAILED DESCRIPTION OF EMBODIMENTS

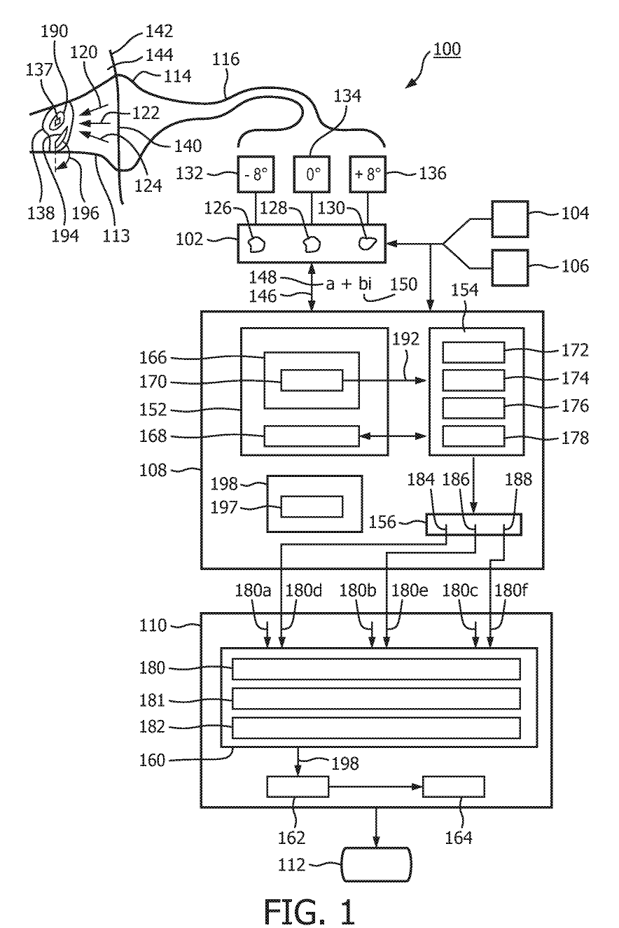

FIG. 1 depicts, by way of illustrative and non-limitative example, a locally-adaptive pixel-compounding apparatus 100. It includes an imaging acquisition module 102, a retrospective dynamic transmit (RDT) focusing module 104 and/or an incoherent RDT focusing module 106, a pixel processor 108, and image processor 110, an imaging display 112, and an imaging probe 114 connected by a cable 116 to the imaging acquisition module 102.

From echo data returning from a transmit beam 113, imaging acquired via the imaging probe 114 is electronically steered into angled views 120, 122, 124 that constitute respective pixel-based images 126, 128, 130 at respective viewing angles 132, 134, 136. The latter are represented in FIG. 1 as, for instance, −8°, 0°, and +8°. Different anglings and a different number of images may be utilized. A pixel 137 is volumetric, i.e., a voxel, and is within one of the three volumetric images 126-130. Pixel 137 coincides spatially with a particular pixel of each of the remaining volumetric images, and coincides spatially with a pixel of a compounded image to be formed. As an alternative to volumetric processing, the images 126-130 are two-dimensional, such as sector scans, and made up of non-volumetric pixels. Here, the differently angled views 120-124 of a region of interest 138 are obtained from a single, acoustic window 140 on an outer surface 142, or skin, of an imaging subject 144, e.g., human patient or animal. Alternatively or in addition, even without electronic steering, a group of views, even uni-directional, can be frequency compounded. Also alternatively or in addition, more than one acoustic window on the outer surface 142 can be utilized for acquiring correspondingly differently angled views. The probe 114 can be moved from window to window, or additional probes are placeable correspondingly at the windows. Temporal compounding of the multiple images is another capability of the apparatus 100.

The pixel processor 108 is configured for receiving channel data 146, a datum of which is represented by a complex number in that is has a nonzero real component 148 and a nonzero imaginary component 150. The pixel processor 108 includes a beamforming module 152, an image content assessment module 154, and a weight determination module 156.

The image processor 110 includes a pixel compounder 160, a logarithmic compression module 162, and a scan conversion module 164.

An electronic steering module 166 and a beamforming summation module 168 are included in the beamforming module 152. The electronic steering module 166 includes a beamforming delay module 170.

The image content assessment module 154 includes a classifier module 172, a coherence factor module 174, a covariance matrix analysis module 176, and a Wiener factor module 178.

The pixel compounder 160 includes a spatial compounder 180, a temporal compounder 181, and a frequency compounder 182. Inputs to the pixel compounder 160 include pixels 180a, 180b, 180c, of the three images 126-130, that spatially correspond to the current pixel of the compound image to be formed, i.e., the current compound image pixel. These inputs are accompanied by inputs 180d, 180e, 180f for respective weights 184, 186, 188 determined by the weight determination module 156. Each of the weights 184-186 may be particular to a single respective pixel 180a, 180b, 180c from among those that mutually spatially correspond. Or each weight 184-188 may serve as an overall weight for application to a group 190 of adjacent pixels in an image from among the three images 126-130, that group being coincident with the adjacent pixels that make up a set of pixels in a compound image to be formed. Output of the pixel compounder 160 is a pixel 191 of a compounded image being formed.

The coherence factor module 174 and covariance matrix analysis module 176 are based on the following principles.

With regard to coherence estimation, let S(m, n, tx, rx) denote complex RF, beamforming-delayed channel data 192, i.e., after applying beamforming delays but before beamsumming. Here, m is the imaging depth/time counter or index, n the channel index, tx the transmit beam index, and rx the receive beam index. A coherence factor (CF) or “focusing criterion” at a pixel (m, rx), or field point, 137 with a single transmit beam is:

$${CF}_{0} ( m , rx ) ≡ \frac{{\vert {\sum }_{n = 1}^{N} S ( m , n , rx , rx ) \vert}^{2}}{N {\sum }_{n = 1}^{N} {\vert S ( m , n , rx , rx ) \vert}^{2}} = \frac{{\vert \frac{1}{N} {\sum }_{n = 1}^{N} S ( m , n , rx , rx ) \vert}^{2}}{\frac{1}{N} {\sum }_{n = 1}^{N} {\vert S ( m , n , rx , rx ) \vert}^{2}} ,$$

where N is the number of channels. The term

$${\vert \frac{1}{N} {\sum }_{n = 1}^{N} S ( m , n , rx , rx ) \vert}^{2}$$

is denoted as I_{c}(m, rx), where the subscript “c” stands for coherent, as it can be interpreted as the average coherent intensity over channels at the point (m, rx). The denominator on the right can be expressed as

$$\frac{1}{N} {\sum }_{n = 1}^{N} {\vert S ( m , n , rx , rx ) \vert}^{2} = \frac{1}{N} {\sum }_{n = 1}^{N} {\vert Δ S ( m , n , rx , rx ) \vert}^{2} + {\vert \frac{1}{N} {\sum }_{n = 1}^{N} S ( m , n , rx , rx ) \vert}^{2}$$

$$where$$

$$Δ S ( m , n , rx , rx ) = S ( m , n , rx , rx ) - \frac{1}{N} {\sum }_{n = 1}^{N} S ( m , n , rx , rx ) .$$

The term

$$\frac{1}{N} {\sum }_{n = 1}^{N} {\vert Δ S ( m , n , rx , rx ) \vert}^{2}$$

is denoted as I_{inc}(m, rx), where the subscript “inc” stands for incoherent. This is because I_{inc}(m, rx) reflects the average intensity of incoherent signals (in the surroundings of (m, rx) decided by the focusing quality on transmit) and is zero when the channel data 144 are fully coherent. Substituting terms,

$${CF}_{0} ( m , rx ) = \frac{{I}_{c} ( m , rx )}{{I}_{inc} ( m , rx ) + {I}_{c} ( m , rx )} = \frac{1}{\frac{{I}_{inc} ( m , rx )}{{I}_{c} ( m , rx )} + 1} .$$

Therefore, CF₀(m, rx) indicates how much the point (m, rx) is brighter than its surroundings. CF₀ ranges between 0 and 1 and it reaches the maximum 1 if and only if the delayed channel data 192 are fully coherent. Full coherence means that S(m, 1, rx, rx)=S(m, 2, rx, rx)==S(m, N, rx, rx). Around a strong point target or a reflector, the CF₀ value is high.

If multiple transmit beams are incorporated into CF estimation, CF is redefinable as:

$$CF ( m , rx ) ≡ \frac{{\sum }_{tx = rx - Δ}^{rx + Δ} {\vert {\sum }_{n = 1}^{N} S ( m , n , tx , rx ) \vert}^{2}}{{\sum }_{tx = rx - Δ}^{rx + Δ} N {\sum }_{n = 1}^{N} {\vert S ( m , n , tx , rx ) \vert}^{2}} \qquad ( definition 1 )$$

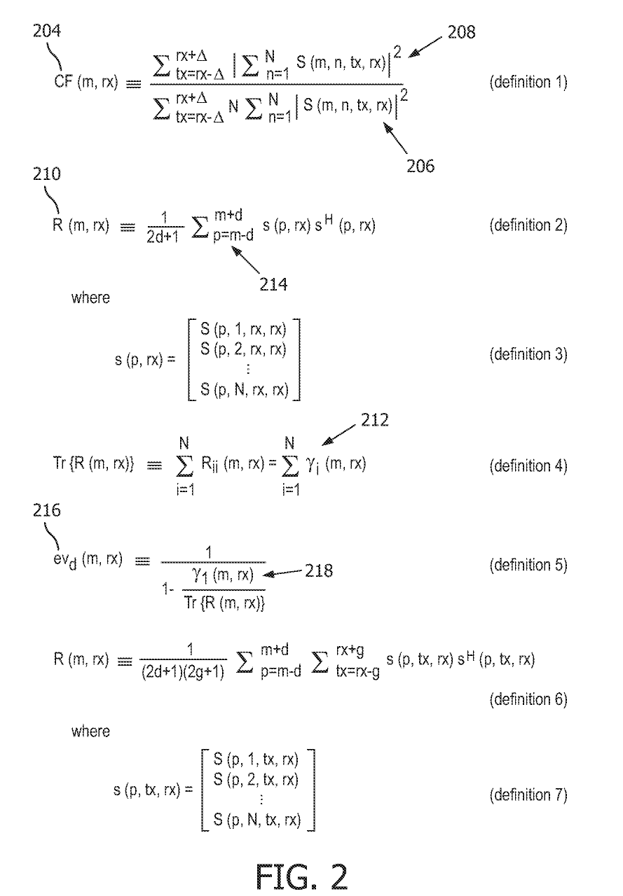

which definition, like the ones that follow, is repeated in FIG. 2. The assessing of local image content with respect to (m, rx) by computing CF(m, rx) commences operating on the delayed channel data 192 no later than upon the beamforming, i.e., the summation Σₙ₌₁^{N} S(m, n, tx, rx).

As mentioned above, the pixel (m, rx) 137 is a function of both an associated receive beam rx and a spatial depth or time. The estimating operates on the delayed channel data 192 by summing, thereby performing beamforming. The CF(m, rx) estimate, or result of the estimating, 204 includes spatial compounding of the CF by summing, over multiple transmit beams, a squared-magnitude function 206 and a squared beamsum 208, i.e. summed result of beamforming. The function 206 and beamsum 208 are both formed by summing over the channels.

Referring now to the covariance matrix analysis, let R(m, rx) denote a covariance matrix, or “correlation/covariance matrix”, 210 at the point (m, rx) obtained by temporal averaging over a range 214 of time or spatial depth:

$$\begin{matrix}R ( m , rx ) ≡ \frac{1}{2 d + 1} {\sum }_{p = m - d}^{m + d} s ( p , rx ) {s}^{H} ( p , rx ) \\ where & ( definition 2 ) \\ s ( p , rx ) = [ \begin{matrix}S ( p , 1 , rx , rx ) \\ S ( p , 2 , rx , rx ) \\ ⋮ \\ S ( p , N , rx , rx )\end{matrix} ] . & ( definition 3 )\end{matrix}$$

As R(m, rx) is positive semidefinite, all of its eigenvalues 212 are real and nonnegative. Denote the eigenvalues by {yᵢ(m, rx)}ᵢ₌₁^{N} with γᵢ≧γᵢ₊₁. Then the trace of R(m, rx)

$$Tr \{ R ( m , rx ) \} ≡ {\sum }_{i = 1}^{N} {R}_{ii} ( m , rx ) = {\sum }_{i = 1}^{N} {γ}_{i} ( m , rx ) . \qquad ( definition 4 )$$

The dominance 216 of the first eigenvalue 218 is represented as

$${ev}_{d} ( m , rx ) ≡ \frac{1}{1 - \frac{{γ}_{1} ( m , rx )}{Tr \{ R ( m , rx ) \}}} . \qquad ( definition 5 )$$

It is infinite if γᵢ(m, rx)=0 for i≧2 (i.e., if the rank of R(m, rx) is 1) as Tr{R(m, rx)}=γ₁(m, rx), and finite otherwise. Summing over several transmits (beam averaging) could also be applied in correlation matrix analysis, as follows:

$$\begin{matrix}R ( m , rx ) ≡ \frac{1}{( 2 d + 1 ) ( 2 g + 1 )} {\sum }_{p = m - d}^{m + d} {\sum }_{tx = rx - g}^{rx + g} s ( p , tx , rx ) {s}^{H} ( p , tx , rx ) & ( definition 6 ) \\ where & \\ s ( p , tx , rx ) = [ \begin{matrix}S ( p , 1 , tx , rx ) \\ S ( p , 2 , tx , rx ) \\ ⋮ \\ S ( p , N , tx , rx )\end{matrix} ] & ( definition 7 )\end{matrix}$$

Another way of combining transmits is to form the covariance matrix from data generated by an algorithm that recreates focused transmit beams retrospectively. An example utilizing RDT focusing is as follows, and, for other such algorithms such as IDRT, plane wave imaging and synthetic aperture beamforming, analogous eigenvalue dominance computations apply:

$$R ( m , rx ) ≡ \frac{1}{2 d + 1} {\sum }_{p = m - d}^{m + d} {s}_{RDT} ( p , rx ) {s}_{RDT}^{H} ( p , rx )$$

$$where$$

$${s}_{RDT} ( p , rx ) = [ \begin{matrix}{S}_{RDT} ( p , 1 , rx ) \\ {S}_{RDT} ( p , 2 , rx ) \\ ⋮ \\ {S}_{RDT} ( p , N , rx )\end{matrix} ] ,$$

and S_{RDT}(p, n, rx) are the dynamically transmit-beamformed complex RF channel data obtained by performing retrospective dynamic transmit (RDT) focusing on the original channel data S(m, n, tx, rx). See U.S. Pat. No. 8,317,712 to Burcher et al. The assessing of local image content with respect to (m, rx) by computing R(m, rx) commences operating on the delayed channel data 192 no later than upon the beamforming, i.e., the summation S_{RDT}(p, rx)s_{RDT}^{H}(p, rx).

In the above bifurcated approach, CF₀(m, rx) or CF(m, rx) can, as with the dominance, likewise be obtained by temporal averaging over a range 214 of time or spatial depth 140.

According to J. R. Robert and M. Fink, “Green's function estimation in speckle using the decomposition of the time reversal operator: Application to aberration correction in medical imaging,” J. Acoust. Soc. Am., vol. 123, no. 2, pp. 866-877, 2008, the dominance of the first eigenvalue ev_{d}(m, rx) can be approximated by 1/(1−CF₁(m, rx)), where CF₁(m, rx) is a coherence factor obtained from channel data S(m, n, tx, rx). Temporal averaging 230, averaging over multiple transmit beams 116, 118, and/or RDT can be applied in calculating CF₁(m, rx). Inversely, coherence factor can be approximated by eigenvalue dominance derived with proper averaging.

In addition to the CF metric and eigenvalue dominance metric, another example of a signal metric is the Wiener factor which is applicable in the case of RDT and IRDT. The Wiener factor module 178 for deriving the Wiener factor is based on the following principles.

In order to compute the Wiener factor corresponding to pixel 137, the following steps are taken:

1) K ultrasound wavefronts (transmits) sequentially insonify the medium. The waves backscattered by the medium are recorded by the array and beamformed in receive to focus on the same pixel 137. It is assumed here that the pixel is formed by RDT, or IRDT, focusing. See U.S. Pat. No. 8,317,712 to Burcher et al. and U.S. Pat. No. 8,317,704 to Robert et al., respectively, both patents being incorporated herein by reference in their entirety.

2) The result is a set of K “receive vectors” rᵢ(P) (i=1 . . . K) of size N samples (one sample per array element) that correspond to a signal coming from pixel 137. Each of the vectors can be seen as a different observation of the pixel 137. The entries of rᵢ(P) are complex, such that the processing is designed to handle a number having, as nonzero, both a real component and an imaginary component.

3) Each of the receive vectors is weighted (by the apodization vector a, which is usually a Box, or Hamming/Hanning, or Riesz window) and summed across the receive elements. This yields K beam-sum values that correspond to the Sample Values (SV) as obtained with the K different insonifications:

{SV₁(P)=a^{H}r₁(P); SV₂(P)=a^{H}r₂(P); . . . SV_{K}(P)=a^{H}r_{K}(P)} (expression 1)

The collection of these K sample values is called the “RDT vector.” Note that the RDT sample value is obtained by summing the values of the RDT vector:

SV_{RDT}=Σᵢ₌₁^{K} a^{H}rᵢ(P) (expression 2)

The Wiener factor is:

$${w}_{wiener} ( P ) = \frac{{\vert {\sum }_{i = 1}^{K} {a}^{H} {r}_{i} ( P ) \vert}^{2}}{{\sum }_{i = 1}^{k} {\vert {a}^{H} {r}_{i} ( P ) \vert}^{2}} \qquad ( expression 3 )$$

The numerator is the square of the coherent sum of the elements of the RDT vector, in other words the RDT sample value squared. The denominator is the incoherent sum of the squared elements of the RDT vector. In other words, if one defines the incoherent RDT sample value (SV_{IRDT}) as the square root of the numerator, then

$${w}_{wiener} ( P ) = \frac{{\vert {SV}_{RDT} ( P ) \vert}^{2}}{{\vert {SV}_{IRDT} ( P ) \vert}^{2}}$$

The Wiener factor is the ratio between the coherent RDT energy and the incoherent RDT energy. It is thus a coherence factor in beam space. It is usable as a signal metric for RDT and IRDT focusing. The assessing of local image content with respect to pixel 137 by computing w_{wiener}(P) commences operating on the receive vectors rᵢ(P) no later than upon the beamforming, i.e., the summation Σᵢ₌₁^{K} a^{H}rᵢ(P).

Image metrics can also be used in lieu of the signal-based coherence factor. For example, known confidence metrics in the literature are usually based on the local gradient and Laplacian of the image. See, for example, Frangi et al, “Multiscale vessel enhancement filtering”, MICCAI 1998). A “confidence factor” is computable from the pre-compressed data as follows: at each pixel, a rectangular box of approximately 20 by 1 pixels is rotated with the spatially corresponding pixel 180a-180c in the middle of the box. The box is rotated from 0 to 170 degrees by increments of 10 degrees. For each orientation of the box, the metric pixel value/mean pixel values inside the box is recorded. The final metric is equal to the maximum of this metric across all angles. Thus the “confidence factor” derived this way takes high values whenever there is sharp contrast between the point of interest and its surroundings, at a given angle. Although assessing performed by the confidence factor computation precedes processing in the compression module 162, it occurs after the beamforming stage rather than at or upon that stage.

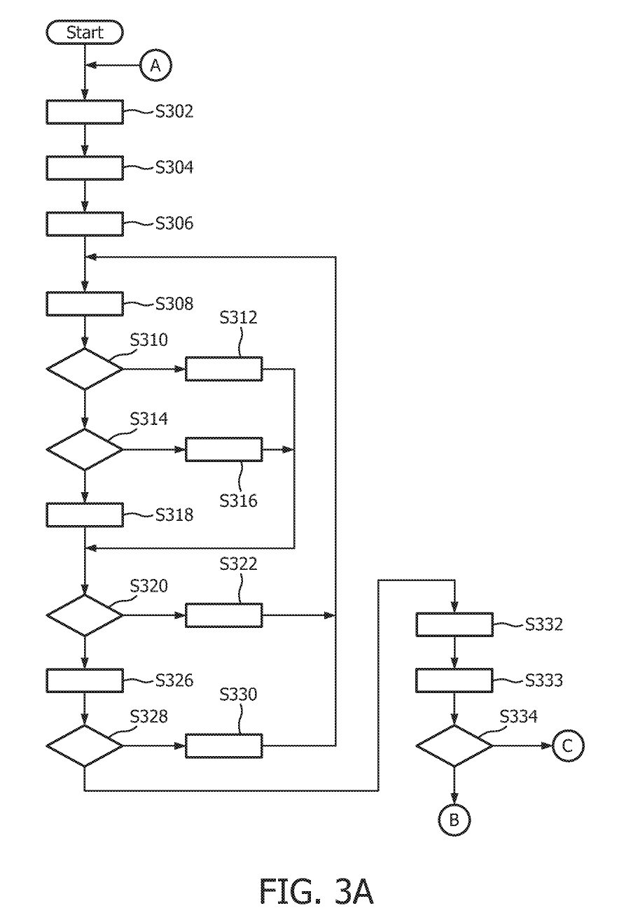

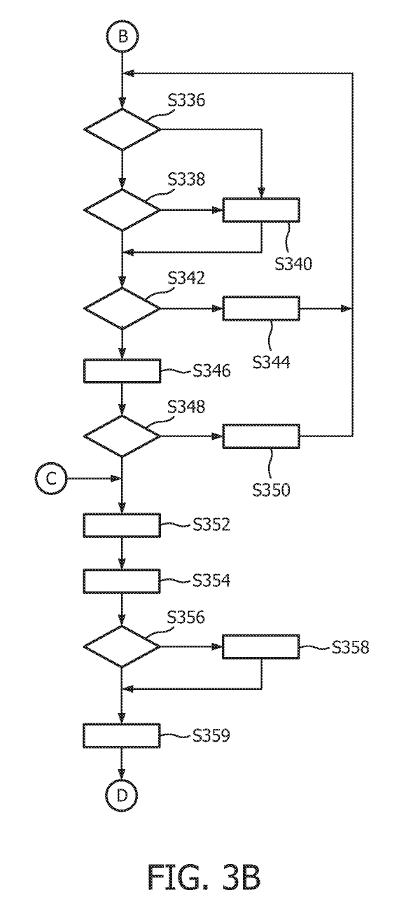

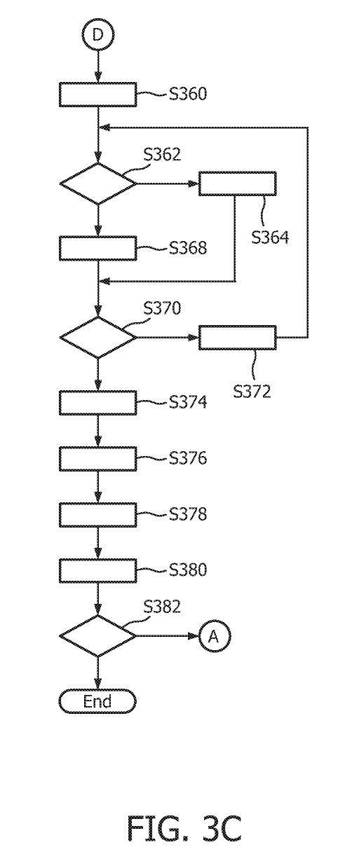

FIGS. 3A through 3C are flow charts exemplary of the signal-metric-based, locally-adaptive pixel-compounding proposed herein.

With reference to FIG. 3A, an image 126-130 is correspondingly acquired, by the imaging acquisition module 102, from each viewing angles 132, 134, 136 (step S302). Processing points to the first pixel 191 of a compounded image to be formed, and to the spatially corresponding pixels 180a-180c of the angle-oriented images 126-130 (step S304). Processing also points to a first angle 132-136 (step S306). The beamforming delay module 170 receives the complex channel data 146 derived from a receive aperture used for receive beamforming the first pixel 191, and applies channel-specific delays to yield the beamforming-delayed channel data 192 (step S308). If RDT and/or IRDT focusing is to be performed (step S310), the Wiener factor module 178 operates upon the beamforming-delayed channel data 192, in the manner discussed herein above, to derive the Wiener factor (step S312). In the apparatus 100, RDT and/or IRDT focusing, or neither, is implemented. If neither RDT nor IRDT focusing is to be performed (step S310), but a coherence factor metric is to be calculated (step S314), the coherence factor module 174 operates upon the beamforming-delayed channel data 192 to calculate a coherence factor (step S316). If neither the Wiener factor nor a coherence factor is to be calculated (step S314), the covariance matrix analysis module 176 operates upon the beamforming-delayed channel data 192 to calculate the dominance of the first eigenvalue of a channel covariance matrix (step S318). After the signal metric is computed, if there exists a next angled view 120-124 (step S320), processing points to that next angle (step S322), and return is made to the delay-applying step S308. If there does not exist a next angled view 120-124 (step S320), the angle counter is reset (step S326) and query is made as to whether there exists a next pixel 191 to process in the current view (step S328). If there is a next pixel 191 (step S328), processing is updated to that next pixel (step S330). Otherwise, if there is no next pixel 191 (step S328), processing again, as in step S304, points to the first pixel 191 of the compounded image to be formed, and to the spatially corresponding pixels 180a-180c of the angle-oriented images 126-130 (step S332). The angle counter is reset (step S333). If classifying of the local information content is implemented (step S334), query is made, as seen from FIG. 3B, as to whether a predetermined feature 194 is detected locally, with respect to the current pixel 191, in the current image 126-130 (step S336). The local information content is searchable for this purpose within any given spatial range, e.g., the 124 pixels of a cube centered on the current pixel 191. If the feature 194 is not detected locally (step S336), query is made as to whether a predetermined orientation 196 is detected locally, with respect to the current pixel 191, in the current image 126-130 (step S338). An example of an image classifier for detecting a feature, such as tubularity, or orientation is disclosed in U.S. Patent Publication No. 2006/0173324 to Cohen-Bacrie et al., the entire disclosure of which is incorporated herein by reference. If either the feature 194 or the orientation 196 is detected (steps S336, S338), the current pixel 191 is marked as important for purposes of weighting in the compounding (step S340). In any event, if a next angle 132-136 exists (step S342), processing points to that next angle (step S344), and return is made to step S336. Otherwise, if a next angle 132-136 does not exist (step S342), the angle counter is reset (step S346). If a next pixel 191 exists (step S348), processing points to that next pixel (step S350). Otherwise, if no next pixel 191 exists (step S348), or if classifying data is not implemented, as seen from step S334, a brightness map is made of the angle-wise maximum brightness pixel-by-pixel (step S352). In other words, over all pixel-based images 126, 128, 130 at respective viewing angles 132, 134, 136, and for a given pixel location, the pixel of maximum brightness is selected. The brightness of the selected pixel is supplied to that given pixel location on the map. This is repeated pixel-location by pixel-location until the map is filled. The map constitutes an image that enhances the visibility of anisotropic structures. However, tissue smearing is maximized and contrast is deteriorated. A map is also made of the angle-wise mean brightness pixel-by-pixel (step S354). By giving equal weight to all views 120-124, the benefits of smoothing out speckle areas are realized. If a minimum map is to be made (step S356), it is made up of the angle-wise minimum brightness pixel-by-pixel (step S358). This image depicts anisotropic structures poorly, but advantageously yields the low brightness values inside cysts. An objective is to not enhance cyst areas, and not to bring sidelobe clutter into cysts. A signal-metric map is also made of the angle-wise maximum coherence factor pixel-by-pixel (step S359). In an alternative implementation, a similar pixel-by-pixel map can instead be based on image metric values. The values for the signal-metric map are normalized by their maximum value, thereby causing the map values to fully occupy the range from zero to one. This step is necessary to re-scale the metric depending on the amount of aberration that may be present in a given acquisition. Optionally the signal-metric map can be processed by, for example, smoothing (ideally with a spatial average of a few resolution cells) or adaptive smoothing such as in the Lee Filters or other algorithms known in the art. Instead of coherence factor, any other signal metric is usable, and an image metric can optionally be additionally used in the weighted compounding that is described herein below. In fact, the classification criterion is, as will be demonstrated herein below, an example of the additional use of an image metric. Referring now to FIG. 3C, processing points to the first pixel 191 of the compounded image to be formed (step S360). If any of the spatially corresponding pixels 180a-180c of the angle-oriented images 126-130 was marked as important is step S340 (step S362), a weighted average is assigned, with a weight of unity for a spatially corresponding pixel 180a-180c that was marked important and with zero being assigned to the remaining spatially corresponding pixels 180a-180c of the current first pixel (step S364). Alternatively, the marking in step S340 may differentiate between found features 194 and found orientations 196, giving, for example, more importance or priority, to features. Another alternative is to split the weighted average between two pixels 180a-180c that were marked important. Also, marking of importance may, instead of garnering the full weight of unity, be accorded a high weight such as 0.75, with signal metric analysis, or other image metric results, affecting the weighting for the other spatially corresponding pixels. If, however, none of the spatially corresponding pixels 180a-180c of the angle-oriented images 126-130 was marked as important is step S340 (step S362), weights are computed as an average, and as a function of the brightness maps and the signal metric map of steps S352-S359 (step S368). Exemplary implementations based on the coherence factor (CF) are discussed herein below. More generally, the objective is now to, based on the signal-metric map, decide which weight to give to the minimum, mean and maximum spatially corresponding pixels 180a-180c to form a final composite image, i.e., compounded image to be formed, that contains all structures with maximum visibility and all cysts with maximum contrast.

Two possible implementations are demonstrated, one which uses the minimum image and another that doesn't. Using the minimum image increases image contrast by decreasing cyst clutter but may also result in unwanted signal reduction from real structures.

In a first implementation, a pixel-wise weighted average is taken of the mean and maximum images. The three rules are: 1) when the CF is above a given threshold tₘₐₓ, select the pixel from the maximum image; 2) when the CF is below a given threshold tₘᵢₙ, select the pixel from the mean image; and 3) in between, combine the two pixels. This can be formalized mathematically as follows:

Normalize CF between tₘᵢₙ and tₘₐₓ:

$${CF}_{norm} = max ( 0 , min ( \frac{CF - {t}_{min}}{{t}_{max} - {t}_{min}} , 1 ) )$$

Determine the weights based on the normalized CF:

wₘₑₐₙ=1−CFₙₒᵣₘ; wₘₐₓ=CFₙₒᵣₘ

Accordingly, instead of compounding the acquired images 126-130 directly, each composite pixel 191 is the weighted average of its counterpart in the brightness map which is made of the angle-wise mean brightness pixel-by-pixel and its counterpart in the brightness map which is made of the angle-wise maximum brightness pixel-by-pixel, those two counterpart pixels being weighted respectively by wₘₑₐₙ and wₘₐₓ. The weights=f(CF) could also have a quadratic, polynomial, or exponential expression.

A second implementation finds the pixel-wise weighted average of the minimum, mean and maximum images. The three rules are: 1) when the CF is above a given threshold tₘₐₓ, select the pixel from the maximum image; 2) when the CF is below a given threshold tₘᵢₙ, select the pixel from the minimum image; and 3) in between, combine the pixels from the minimum, mean and maximum images, although some potential value of CF will exclusively select the pixel from the mean image.

This can be formalized mathematically as follows:

Normalize CF between tₘᵢₙ and tₘₐₓ:

$${CF}_{norm} = max ( 0 , min ( \frac{CF - {t}_{min}}{{t}_{max} - {t}_{min}} , 1 ) )$$

Determine the weights based on the normalized CF:

wₘᵢₙ=(1−CFₙₒᵣₘ)²; wₘₐₓ=(CFₙₒᵣₘ)²; wₘₑₐₙ=1−wₘᵢₙ−wₘₐₓ

The weights=f(CF) could also have a linear, polynomial, or exponential expression.

In either event, i.e., whether or not the above-described classification or a signal metric is used in the weighting, and regardless of a whether additional metrics, signal or image, are used, if a next pixel 191 exists (step S370), processing points to that next pixel (step S372) and processing returns to step S362. If, on the other hand, no next pixel 192 remains (step S370), the weights are applied pixel-by-pixel to form weighted pixels, the weighted pixels being summed to form a weighted average for each pixel 191, these latter pixels collectively constituting the compound image (step S374).

Speckle artifacts introduced by the adaptive method can be removed while retaining the contrast gains as follows. The mean image created in step S354 is subtracted from the compound image created in step S374 (step S376). The resulting difference image is low-pass filtered (step S378). The low-pass-filtered image is added to the mean image to yield a despeckled image (step S380). The low-frequency image changes, such as larger structures and cysts, are consequently retained, while the higher frequency changes, such as speckle increase, are eliminated. The low-pass filter is realizable by convolution with, for example, a Gaussian or box kernel. A composite image is now ready for display.

Alternatively with regard to speckle reduction, a programmable digital filter 197 can be introduced to receive the beamformed data and separate the data of higher spatial frequency, which contain the speckle signal, from the data of lower spatial frequency. In this multi-scale approach, a multi-scale module 198 passes on only the lower-frequency data to the image content assessment module 154 for adaptive compounding. The higher-frequency data are assigned equal compounding weights in the weight determination module 156. Furthermore, different metrics and different formulas for combining compounded sub-views into an image based on the metrics, may be advantageously applied at each subscale. For instance, low spatial frequencies may be more aggressively enhanced than higher frequency subscales.

If image acquisition is to continue (step S382), return is made to step S302.

Optionally, the weights determined in a neighborhood of a spatially corresponding pixel 180a-180c may be combined, such as by averaging. A neighborhood could be a cluster of pixel, centered on the current pixel. In that case, compounding is performed with less granularity, i.e., neighborhood by neighborhood, instead of pixel by pixel.

An image compounding apparatus acquires, via ultrasound, pixel-based images of a region of interest for, by compounding, forming a composite image of the region. The image includes composite pixels that spatially correspond respectively to pixels of the images. Further included is a pixel processor for beamforming with respect to a pixel from among the pixels, and for assessing, with respect to the composite pixel and from the data acquired, amounts of local information content of respective ones of the images. The processor determines, based on the assessment, weights for respective application, in the forming, to the pixels, of the images, that spatially correspond to the composite pixel. In some embodiments, the assessing commences operating on the data no later than upon the beamforming. In some embodiments, brightness values are assigned to the spatially corresponding pixels; and, in spatial correspondence, the maximum and the mean values are determined. They are then utilized in weighting the compounding.

While the invention has been illustrated and described in detail in the drawings and foregoing description, such illustration and description are to be considered illustrative or exemplary and not restrictive; the invention is not limited to the disclosed embodiments.

For example, within the intended scope of what is proposed herein is a computer readable medium, as described below, such as an integrated circuit that embodies a computer program having instructions executable for performing the process represented in FIGS. 3A-3C. The processing is implementable by any combination of software, hardware and firmware.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. Any reference signs in the claims should not be construed as limiting the scope.

A computer program can be stored momentarily, temporarily or for a longer period of time on a suitable computer-readable medium, such as an optical storage medium or a solid-state medium. Such a medium is non-transitory only in the sense of not being a transitory, propagating signal, but includes other forms of computer-readable media such as register memory, processor cache, RAM and other volatile memory.

A single processor or other unit may fulfill the functions of several items recited in the claims. The mere fact that certain measures are recited in mutually different dependent claims does not indicate that a combination of these measures cannot be used to advantage.

1. A pixel-compounding imaging apparatus, comprising:
an imaging acquisition module configured to acquire multiple pixel-based ultrasound images of a region of interest and to form a compounded image of said region, said compounded image comprising a plurality of pixels that spatially correspond respectively to pixels of said images; and
a pixel processor configured to perform the following steps:
assess, based on data not yet beamformed and with respect to a pixel from among said plurality of pixels, amounts of local information-content contained within each of the multiple pixel-based ultrasound images; and
determine weights to be applied to the plurality of pixels that spatially correspond to said pixels of said multiple pixel-based ultrasound images.

2. The apparatus of claim 1, wherein said data has been subject to beamforming delays.

3. (canceled)

4. The apparatus of claim 1, said region of interest residing within an imaging subject having an outer surface, said apparatus further comprising an ultrasound imaging probe configured to acquire said multiple pixel-based ultrasound images from a single ultrasound acoustic window on said surface, said multiple pixel-based ultrasound images respectively being differently angled views of said region of interest.

5. The apparatus of claim 1, being configured to form the compounded image by temporal compounding.

6. (canceled)

7. (canceled)

8. The apparatus of claim 1, said application forming summands of a weighted average, said weights being functionally related to the assessed amounts.

9. (canceled)

10. The apparatus of claim 1, wherein said data comprises channel data and the access step comprises assessing coherence of said channel data.

11. The apparatus of claim 1, wherein said data comprises channel data and the assess step comprises calculating dominance of an eigenvalue of a covariance matrix that represents covariance of said channel data.

12. The apparatus of claim 1, being configured to perform at least one of retrospective dynamic transmit (RDT) focusing, and incoherent RDT focusing, in forming a pixel from among said pixels that spatially correspond and to which a weight from among said weights is applied.

13. (canceled)

14. The apparatus of claim 1, being configured to assign brightness values respectively to said plurality of pixels, and to use a maximum from among said brightness values in said determining for multiple ones of said weights.

15. The apparatus of claim 14, being configured to identify a minimum from among said brightness values, and use the identified minimum in said determining for multiple ones of said weights.

16.-21.

22. The apparatus of claim 1, being configured to average the spatially corresponding images, pixel by pixel, to yield an average image, apply a low-pass filter to produce a difference between said average image and said image of said region, and add the difference to said average image.

23. (canceled)

24. (canceled)

25. The apparatus of claim 1, wherein the assess step comprises utilizing at least one metric from the group consisting of a coherence factor, dominance of a first eigenvalue, and a Weiner factor.

26. The apparatus of claim 1 wherein said weights are functionally related to the amounts of local information content.
